(19) United States
(12) Patent Application Publication
(10) Pub. No.: US 2005/0031669 A1
(43) Pub. Date: Feb. 10, 2005

(54) Rate controlled release of a pharmaceutical agent in a biodegradable device

(75) Inventors: Afshin Shafiee, Rochester, NY (US); Joseph C. Salamone, Fairport, NY (US); Dharmendra Jani, Fairport, NY (US); Stephen Paul Bartels, Pittsford, NY (US); Jay F. Kunzler, Canandaigua, NY (US)

Correspondence Address: Bausch & Lomb Incorporated, One Bausch & Lomb Place, Rochester, NY, 14604-2701, US

(73) Assignee: Bausch & Lomb Incorporated, Rochester, NY (US)

(21) Appl. No.: 10/887,381

(22) Filed: Jul. 8, 2004

Related U.S. Application Data
(63) Continuation of application No. 10/462,184, filed on Jun. 16, 2003.

(51) Int. Cl. C12N 5/02; A61K 9/00

(52) U.S. Cl. 424/426

(57) ABSTRACT

Chemical erosion drug delivery systems are provided that allow sustained release of therapeutic agents within a treated area for a prolonged period of time.

### RELATED APPLICATIONS

This application is a Continuation-In-Part of U.S. patent application Ser. No. 10/462,184, filed Jun. 16, 2003.

### FIELD OF THE INVENTION

The present invention relates to the field of drug delivery and more particular to the field of drug delivery from a biodegradable drug delivery device.

### BACKGROUND OF THE INVENTION

Conventional drug delivery involving frequent periodic dosing is not ideal or practical in many instances. For example, with more toxic drugs, conventional periodic dosing can result in high initial drug levels at the time of dosing, followed by low drug levels between doses often times below levels of therapeutic value. Likewise, conventional periodic dosing may not be practical or therapeutically effective in certain instances such as with pharmaceutical therapies targeting the inner eye or brain, due to inner eye and brain blood barriers.

During the last two decades, significant advances have been made in the design of controlled release drug delivery systems. Such advances have been made in an attempt to overcome some of the drug delivery shortcomings noted above. In general, controlled release drug delivery systems include both sustained drug delivery systems designed to deliver a drug for a predetermined period of time, and targeted drug delivery systems designed to deliver a drug to a specific area or organ of the body. Sustained and/or targeted controlled release drug delivery systems may vary considerably by mode of drug release within three basic drug controlled release categories. Basic drug controlled release categories include diffusion controlled release, chemical erosion controlled release and solvent activation controlled release. In a diffusion controlled release drug delivery system, a drug is surrounded by an inert barrier and diffuses from an inner reservoir, or a drug is dispersed throughout a non-biodegradable polymer and diffuses from the polymer matrix. In a chemical erosion controlled release drug delivery system, a drug is distributed throughout a biodegradable polymer. The biodegradable polymer is designed to degrade as a result of hydrolysis to then release the drug. In a solvent activation controlled release drug delivery system, a drug is immobilized on polymers within a drug delivery system. Upon solvent activation, the solvent sensitive polymer degrades or swells to release the drug.

The drug release rate from a drug delivery system is typically manipulated through the selection of the biodegradable polymer(s) employed in the system. Biodegradable polymers have varying rates of hydrolytic ability based on the polymers' molecular weights and copolymer ratios, e.g., lactic acid to glycolic acid (LA:GA). The greater the hydrolytic ability of the biodegradable polymer, the greater the drug release rate. The lesser the hydrolytic ability of the biodegradable polymer, the lesser the drug release rate.

U.S. Pat. No. 5,869,079 teaches a drug delivery system using biodegradable polymers, such as a polyester of lactic acid and glycolic acid mixed with one or more active agents. Modifiers having a higher solubility were added to low solubility active agents to increase the rate of drug delivery. Modifiers having a lower solubility were mixed with relatively high soluble active agents to decrease the rate of drug delivery. Adding modifiers increases the weight of a delivery device. It would be desirable if the release rate could be modified without adding additional weight to the drug delivery device or system. It would be further desirable that a drug delivery device has a high a concentration of active agent as possible while obtaining a desired drug delivery profile. It is desired in one embodiment to have a drug that can be delivered in a therapeutically effective amount over a longer period of time.

U.S. Pat. No. 6,726,918 teaches a drug delivery system using biodegradable polymers, such as a polyester of lactic acid and glycolic acid mixed with one or more active agents. A delivery profile is described where a steroidal anti-inflammatory agent is delivered in an amount to reach a concentration equivalent to at least about 0.05 μg/ml concentration of dexamethasone within 48 hours and at least about 0.03 μg/ml for a period of three weeks.

Example 1 tested in vitro the release rate of a biodegradable implant comprising 70:30 ratio of dexamethasone to a polymer comprising 1 part lactic acid to 1 part glycolic acid. Example 6 tested the release rate of a biodegradable implant comprising a 50:50 ratio of dexamethasone to a polymer comprising 1 part lactic acid to 1 part glycolic acid. The 40% increase in dexamethasone in the device of Example 1 compared to the device of Example 6 resulted in a shorter duration of delivery and approximately 75% increase in the release rate for the first seven days. It would be desirable to formulate a drug delivery device that had a lower release rate and an extended duration of release.

Furthermore, because of the shortcomings of conventional drug delivery noted above, a need exists for methods of controlled release drug delivery systems that allow for manipulation and control of drug release rates depending on the drug to be delivered, the location of delivery, the purpose of delivery and/or the therapeutic requirements of the individual patient.

### SUMMARY OF THE INVENTION

The present invention comprises a chemical erosion controlled drug delivery system or device that comprises a mixture or matrix of a biodegradable polymer and a hydrophobic pharmaceutically-active agent in a therapeutically effective amount. In one embodiment, the drug delivery system or device has a selected concentration of the pharmaceutically-active agent such that when the drug delivery system or device is compared to a comparative system or device with an incrementally lower concentration of the pharmaceutically-active agent, the drug delivery system or device has a release rate for the pharmaceutically-active agent that is less than proportionally higher, the same or lower than a comparative system or device.

In yet another embodiment, the drug delivery system or device has a selected concentration of the pharmaceutically-active agent such that when the drug delivery system or device is compared to a comparative system or device with an incrementally lower concentration of the pharmaceutically-active agent, the drug delivery system or device has a duration of release of the pharmaceutically-active agent that is the same or longer than the comparative system or device.

In one embodiment, the drug delivery system or device has a selected concentration of the pharmaceutically-active agent such that when the drug delivery system or device is compared to a comparative system with an incrementally lower concentration of the pharmaceutically-active agent, the drug delivery system or device (i) has a release rate for the pharmaceutically-active agent that is less than proportionally higher, the same or lower than a comparative system or device and/or (ii) has a duration of release of the pharmaceutically-active agent that is the same or longer than the comparative system or device.

In another embodiment, there is a chemical erosion controlled drug delivery system comprising:
a biodegradable polymer; and
a hydrophobic pharmaceutically-active agent selected from the group consisting of ametantrone, amphotericin B, annamycin, cyclosporin, daunorubicin, diazepam, doxorubicin, elliptinium, etoposide, fluocinolone acetonide, ketoconazole, methotrexate, miconazole, mitoxantrone, nystatin, phenytoin, Iodeprednol, triamcinolone acetonide and vincristine in a therapeutically effective amount. The drug delivery system, of one embodiment, has a selected concentration of the pharmaceutically-active agent such that when the drug delivery system is compared to a comparative system with an incrementally lower concentration of the pharmaceutically-active agent, the drug delivery system (i) has a release rate for the pharmaceutically-active agent that is less than proportionally higher, the same or lower than a comparative system and/or (ii) has a duration of release of the pharmaceutically-active agent that is the same or longer than the comparative system.

In one embodiment, there is a drug delivery device comprising a matrix of a biodegradable polymer and a hydrophobic pharmaceutically-active agent in a therapeutically effective amount. The hydrophobic pharmaceutically-active agent has a solubility that is less than about 90 μg/ml in a buffered saline solution at 25° C.

In another embodiment, there is a chemical erosion controlled drug delivery device comprising: a therapeutic mixture of a biodegradable polymer and a minimum amount of about 45 wt. % of a pharmaceutically-active agent based upon the total weight of the biodegradable polymer and the pharmaceutically-active agent, wherein the pharmaceutically-active agent is characterized in that a 55 wt. % mixture of the pharmaceutically-active agent in a PLGA test matrix releases no more than about 70 wt % of the pharmaceutically-active agent in a three-week period and that the cumulative release rate of the 55 wt. % mixture of the hydrophobic pharmaceutically-active agent in a PLGA test matrix is not more than about 10% greater than the cumulative release rate of a 35 wt. % mixture of the pharmaceutically-active agent in a test matrix over a three-week test period.

### BRIEF DESCRIPTION OF THE DRAWINGS

FIG. 1 is a graphical representation depicting 100 percent 50/50 poly(DL-lactide-co-glycolide) polymer (PLGA) (placebo) implant hydrolysis absorbance values over time;

FIG. 2 is a graphical representation depicting 100 percent 50/50 PLGA (placebo) implant pH over time;

FIG. 3 is a graphical representation depicting drug release rates over time for 35 percent fluocinolone acetonide (FA) implant—Sample 1;

FIG. 4 is a graphical representation depicting drug release rates over time for 35 percent FA implant—Sample 2;

FIG. 5 is a graphical representation depicting drug release rates over time for 35 percent FA implant—Sample 3;

FIG. 6 is a graphical representation depicting the percent cumulative drug release rates over time for 35 percent FA implant—Sample 1;

FIG. 7 is a graphical representation depicting the percent cumulative drug release rates over time for 35 percent FA implant—Sample 2;

FIG. 8 is a graphical representation depicting the percent cumulative drug release rates over time for 35 percent FA implant—Sample 3;

FIG. 9 is a graphical representation depicting 35 percent FA implant, Samples 1, 2 and 3, pH over time;

FIG. 10 is a graphical representation depicting drug release rates over time for 55 percent FA implant—Sample 1;

FIG. 11 is a graphical representation depicting drug release rates over time for 55 percent FA implant—Sample 2;

FIG. 12 is a graphical representation depicting drug release rates over time for 55 percent FA implant—Sample 3;

FIG. 13 is a graphical representation depicting the percent cumulative drug release rates over time for 55 percent FA implant—Sample 1;

FIG. 14 is a graphical representation depicting the percent cumulative drug release rates over time for 55 percent FA implant—Sample 2;

FIG. 15 is a graphical representation depicting the percent cumulative drug release rates over time for 55 percent FA implant—Sample 3;

FIG. 16 is a graphical representation depicting 55 percent FA implant, Samples 1, 2 and 3, pH over time;

FIG. 17 is a graphical representation depicting 35 percent FA implant, Samples 1, 2 and 3, drug release rates and percent cumulative drug release rates over time;

FIG. 18 is a graphical representation depicting 55 percent FA implant, Samples 1, 2 and 3, drug release rates and percent cumulative drug release rates over time; and

FIG. 19 is a graphical representation depicting 35 percent and 55 percent FA implants, drug release rates and percent cumulative drug release rates over 70 days.

### DETAILED DESCRIPTION OF THE INVENTION

The present invention comprises a chemical erosion controlled drug delivery system or device that comprises a mixture or matrix of a biodegradable polymer and a hydrophobic pharmaceutically-active agent in a therapeutically effective amount. In an embodiment, the mixture consists essentially of biodegradable polymer and a therapeutically effective amount of hydrophobic pharmaceutically-active agent.

In yet another embodiment, the drug delivery system or device has a selected concentration of the pharmaceutically-active agent such that when the drug delivery system or device is compared to a comparative system with an incrementally lower concentration of the pharmaceutically-active agent, the drug delivery system or device (i) has a release rate for the pharmaceutically-active agent that is less than proportionally higher, the same or lower than a comparative system or device and/or (ii) has a duration of release of the pharmaceutically-active agent that is the same or longer than the comparative system or device.

The invention in its one or more embodiments can better be understood with reference to one or more of the following definitions:

“Release rate” as it pertains to a pharmaceutically-active agent is defined as the amount of the pharmaceutically-active agent that leaves the system, device, matrix or apparatus in a period of time.

“Comparative system” or “comparative device” is defined as a drug delivery system or drug delivery device that is made for the purpose of determining the effect of a change in the concentration from a selected concentration. The comparative system or comparative device is identical to the drug delivery system to which it is being compared except that the concentration of pharmaceutical agent in the biodegradable polymer of the comparative system relative to the drug delivery system to which it is being compared differs by an amount.

“Chemical erosion controlled drug delivery” is defined as the delivery of a pharmaceutically-active agent at a rate that is proportional to the rate of chemical erosion or dissolution of a polymer resulting from the exposure of the drug delivery to an aqueous medium such as bodily fluids.

“Biodegradable polymer” defined as is a polymer that chemically degrades or dissolves upon contact with an aqueous solution such as bodily fluid.

“Incremental” as defined herein is a step change in an amount of one variable that is sufficient to predict with statistical reliability the marginal response of another variable. By way of example and not by limitation, an incremental increase in concentration of an active agent is an increase in an amount sufficient to determine the response of other variables—for example release rate or duration of release.

“Duration of release” is defined as the duration of time that a drug delivery system or matrix releases 90% of a pharmaceutically-active agent.

“PLGA test matrix” is defined as a polymer containing 50% racemic lactic acid and 50% glycolic acid having an intrinsic viscosity of 0.17. The polymer is prepared by mixing a sample of PLGA polymer powder with a solid form of a pharmaceutically-active agent. The mixture of these components is mixed for a sufficient period of time to ensure a consistent mixture of the polymer and agent. Thereafter, it is extruded at a temperature sufficient to fabricate a filament and typically in the range of from 50° C. to 120° C. The mixture is extruded into 0.5 mm diameter filaments that are cut into desired lengths.

“Less than proportionally” as it pertains to a change in one variable relative to another variable is defined as a less than X % change in the one variable resulting from an X % change in the other variable. By way of example, a one percent increase in one variable resulting from a 1.5% increase in another variable is a less than proportional change in the one variable relative to the other variable. A 1% change in one variable resulting from a 1% change in another variable is not a less than proportional change of the one variable relative to the other variable.

In one embodiment, the incrementally lower concentration is 1% lower than the selected concentration and the drug delivery system (i) has a release rate for the pharmaceutically-active agent that is no more than about 0.9% higher, the same or lower than a comparative system. In another embodiment, the incrementally lower concentration is 1% lower than the selected concentration and the drug delivery system (i) has a release rate for the pharmaceutically-active agent that is no more than about 0.7%, about 0.5% about 0.4%, about 0.3%, or about 0.2% higher, the same or lower than a comparative system.

In an embodiment, the active agent has a selected concentration such that a 1% increase in concentration results in an increase in the duration of release that is a minimum of about 0.1% of one embodiment.

In one embodiment, there is a drug delivery device comprising a matrix of a biodegradable polymer and a hydrophobic pharmaceutically-active agent in a therapeutically effective amount. The hydrophobic pharmaceutically-active agent has a solubility that is less than about 90 μg/ml in a buffered saline solution at 25° C.

In one embodiment, the drug delivery device delivers a minimum of about 0.1 μg is released over a minimum period of 3 weeks. In another embodiment, the drug delivery device delivers a minimum of about 0.5 μg, about 1 μg, about 2 μg, about 5 μg, about 10 μg, about 50 μg, about 100 μg and/or a maximum of about 50 mg, about 25 mg, about 15 mg, about 10 mg, about 5 mg or about 1 mg over a minimum period of about 3 weeks, about 6 weeks, about 12 weeks, about 24 weeks, about 30 weeks, about 36 weeks, about 40 weeks, about 48 weeks or about 52 weeks.

In another embodiment, there is a chemical erosion controlled drug delivery device comprising: a therapeutic mixture of a biodegradable polymer and a minimum amount of about 45 wt. % of a pharmaceutically-active agent based upon the total weight of the biodegradable polymer and the pharmaceutically-active agent, wherein the pharmaceutically-active agent is characterized in that a 55 wt. % mixture of the pharmaceutically-active agent in a PLGA test matrix releases no more than about 70 wt % of the pharmaceutically-active agent in a three-week period and that the cumulative release rate of the 55 wt. % mixture of the hydrophobic pharmaceutically-active agent in a PLGA test matrix is not more than about 10% greater than the cumulative release rate of a 35 wt. % mixture of the pharmaceutically-active agent in a test matrix over a three-week test period.

In one embodiment, the 55 wt. % mixture of the pharmaceutically-active agent in a PLGA test matrix releases no more than about 60 wt % of the pharmaceutically-active agent in a three-week period. Preferably, the 55 wt. % mixture of the pharmaceutically-active agent in a PLGA test matrix releases no more than about 50 wt %, about 40 wt. %, about 30 wt. % or about 20 wt. % of the pharmaceutically-active agent in a three-week period.

In one embodiment, the 55 wt. % mixture of the hydrophobic pharmaceutically-active agent in a PLGA test matrix is not more than about 5% greater than the cumulative release rate of a 35 wt. % mixture of the pharmaceutically-active agent in a test matrix over a three-week test period. In one embodiment, the cumulative release rate of the 55 wt. % mixture of the hydrophobic pharmaceutically-active agent in a PLGA test matrix is not more than the cumulative release rate of a 35 wt. % mixture of the pharmaceutically-active agent in a test matrix over a three-week test period. In another embodiment, the cumulative release rate of the 55 wt. % mixture of the hydrophobic pharmaceutically-active agent in a PLGA test matrix is about 5% less, about 10% less, about 25% less, about 50% less or about 100% less than the cumulative release rate of a 35 wt. % mixture of the pharmaceutically-active agent in a test matrix over a three-week test period.

The drug delivery system of at least one embodiment of the present invention is preferably sized and configured to be inserted into the ocular region of a human patient. Typically, the system is sized and configured to be inserted into the posterior segment of the eye of a human patient—preferably the vitreous of the eye of a human patient.

To fit in the eye of a patient, the system generally occupies a maximum volume of about 26 mm³. Typically, the system occupies a maximum volume of about 15 mm³, about 10 mm³, about 4 mm³ or about 2 mm³. Additionally or alternatively, the system has a maximum mass of about 50 mg. In one embodiment, the system or device has a maximum mass of about 25 mg, about 15 mg, about 10 mg, about 5 mg or about 1 mg.

When formulating a drug delivery system, it is desirable to have a drug delivery system comprise as much pharmaceutically-active agent as is feasible for the particular application. For example, a drug delivery device inserted into the eye requires sufficient biodegradable polymer for sustained release and the overall size must not be too large so as to interfere with the function of the eye. Typically, the system has a maximum amount of the pharmaceutically-active agent of about 25 mg. In one embodiment, the system or device has a maximum amount of the pharmaceutically-active agent of about 10 mg, about 1 mg, about 0.5 mg or about 0.1 mg.

The drug delivery system of one embodiment contains at least one pharmaceutically-active agent that is selected from the group consisting of cytokines, tyrosine kinase inhibitors and steroidal hormones. In another embodiment, at least one pharmaceutically-active agent is selected from the group consisting of anti-glaucoma agents, neuroprotection agents, beta blockers, mitotics, epinephrine, anti-diabetic edema agents, vascular endothelial growth factor (VEGF) antagonists, tyrosine kinase inhibitors, pyrrolyl-methylene-indolinones, C₆₋₄₅ phenyl amino alkoxy quinazolines, anti-proliferative vitreoretinopathy agents, anti-inflammatory agents, immunological response modifiers, anti-ocular angiogenesis agents, anti-mobility agents, steroids, matrix metalloproteinase (MMP) inhibitors, humanized antibodies, aptamers, peptides, antibiotics, angiogenesis targeting agents, anti-cataract and anti-diabetic retinopathy agents, thiol cross-linking agents, anticancer agents, immune modulators, anti-clotting agents, anti-tissue damage agents, proteins, nucleic acids, anti-fibrous agents, non-steroidal anti-inflammatory agents, antibiotics, antipathogens, piperazine derivatives, cycloplegic and mydriatic agents anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

It is desirable that the agent be hydrophobic and have a solubility in water that is less than about 90 μg/ml in a buffered saline solution at 25° C. Typically, the hydrophobic pharmaceutically-active agent has a solubility that is a maximum of about 80 μg/ml, about 70 μg/ml, about 60 μg/ml, about 50 μg/ml, about 40 μg/ml, about 30 μg/ml, about 20 μg/ml, about 10 μg/ml, or about 5 μg/ml.

In one embodiment, the hydrophobic pharmaceutically-active agent is selected from the group consisting of ametantrone, amphotericin B, annamycin, cyclosporin, daunorubicin, diazepam, doxorubicin, elliptinium, etoposide, fluocinolone acetonide, ketoconazole, methotrexate, miconazole, mitoxantrone, nystatin, phenytoin, lodeprednol, triamcinolone acetonide and vincristine.

In one embodiment, the biodegradable polymer is selected from the group consisting of poly(lactide)s, poly(glycolide)s, poly(lactide-co-glycolide)s, poly(lactic acid)s, poly(lactic acid-co-glycolic acid)s, polycaprolactones, polycarbonates, poly(ester amide)s, polyanhydrides, poly(amino acid)s, polyorthoesters, polyacetals, polycyanoacrylates, poly(ether ester)s, polydioxanones, poly(alkylene alkylate)s, copolymers of poly(ethylene glycol) and polyorthoesters, biodegradable polyurethanes and blends and copolymers thereof.

The biodegradable polymer of one embodiment is preferably poly(lactic acid-co-glycolic acid)s. Typically, the drug delivery system has a biodegradable polymer that has a ratio of lactic acid to glycolic acid that is a minimum of 0.1 and a maximum of about 10. Preferably, the ratio of lactic acid to glycolic acid is a minimum of about 0.2, about 0.4, about 0.8, about 0.9 or about 1. Preferably, the ratio of lactic acid to glycolic acid is a maximum of about 10, about 8, about 6, about 4, about 2 or about 1 according to one embodiment.

In one embodiment, the biodegradable polymer has a ratio of poly(lactic-co-glycolic) acid to the pharmaceutically-active agent that is a minimum of about is a minimum of about 0.8 and a maximum of about 4. Preferably, the ratio of poly(lactic-co-glycolic)acid to the pharmaceutically-active agent is a minimum of about 0.2, about 0.9, about 1 about 1.5 or about 2. Preferably, the ratio of lactic acid to glycolic acid is a maximum of about 4, about 3.5, about 3, about 2.5 or about 2.

In one embodiment, there is drug delivery device or system that has a matrix or mixture comprising a pharmaceutically-active agent and a biodegradable polymer. The device or system has a minimum amount of about 50 wt. % of a pharmaceutically-active agent based upon the total weight of the matrix, mixture or amount biodegradable polymer plus amount of the pharmaceutically-active agent.

Typically, the device has a minimum amount of about 50 wt. %, 55 wt. %, 60 wt. % and or a maximum amount of about 80 wt. %, 75 wt. %, 70 wt. %, 65 wt. % or 60 wt. % of a pharmaceutically-active agent based upon the total weight of the biodegradable polymer and the pharmaceutically-active agent.

In another embodiment, the drug delivery system comprises a hydrophobic agent. A hydrophobic agent is a material other than a pharmaceutically-active agent that is added to the matrix of a biodegradable polymer and a hydrophobic pharmaceutically-active agent to enhance the hydrophobicity of the matrix.

Preferably, the hydrophobic agent is selected from the group consisting of glycerol triacetate, glycerol diacetate, diethyl phthalate, dimethyl phthalate, phthalate esters, phosphate esters, fatty acid esters, glycerol derivatives, acetyl triethyl citrate, dibutyl tartrate and combinations thereof. In one embodiment, the hydrophobic agent is selected from the group consisting of glycerol triacetate, glycerol diacetate, diethyl phthalate, dimethyl phthalate, phthalate esters, phosphate esters, fatty acid esters, glycerol derivatives, acetyl triethyl citrate, dibutyl tartrate and combinations thereof.

In one embodiment, the hydrophobic agent has a solubility greater than 90 μg/ml in a buffered saline solution at 25° C. Typically, the hydrophobic agent has a solubility that is a maximum of about 80 μg/ml, about 70 μg/ml, about 60 μg/ml, about 50 μg/ml, about 40 μg/ml, about 30 μg/ml, about 20 μg/ml, about 10 μg/ml, or about 5 μg/ml.

According to one embodiment of the present invention, there is a method of making one or more of the drug delivery systems or devices disclosed herein by encapsulating in a biodegradable polymer a therapeutically effective amount of at least one pharmaceutically-active agent. The drug delivery system or device is sized and configured to be inserted into the eye of a patient.

According to one embodiment of the present invention, there is a method of making one or more of the drug delivery systems or devices disclosed herein by mixing in a biodegradable polymer a therapeutically effective amount of at least one pharmaceutically-active agent. The drug delivery system is sized and configured to be inserted into the eye of a patient. According to another embodiment of the present invention, there is a method of using one or more drug delivery system or device disclosed herein. The method comprises creating an incision within an eye. Thereafter, implanting the system within said eye through said incision—generally using a cannula used along with a needle of a vitrectomy system.

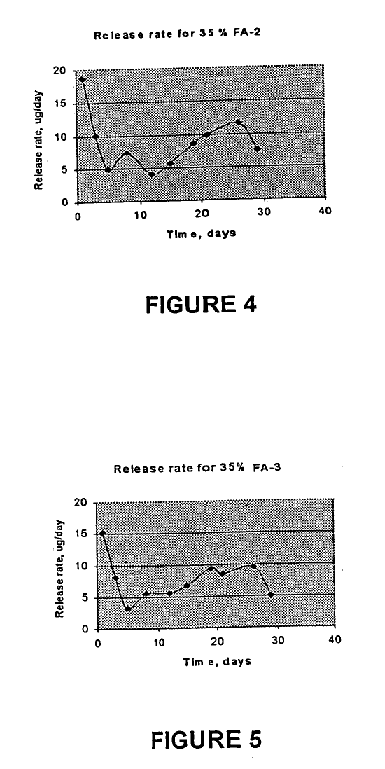

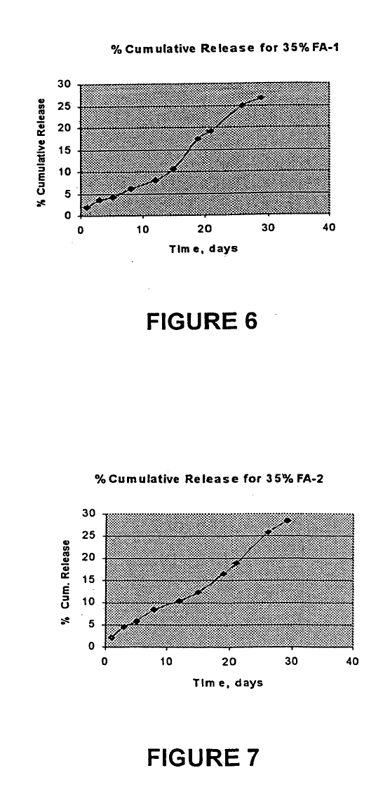

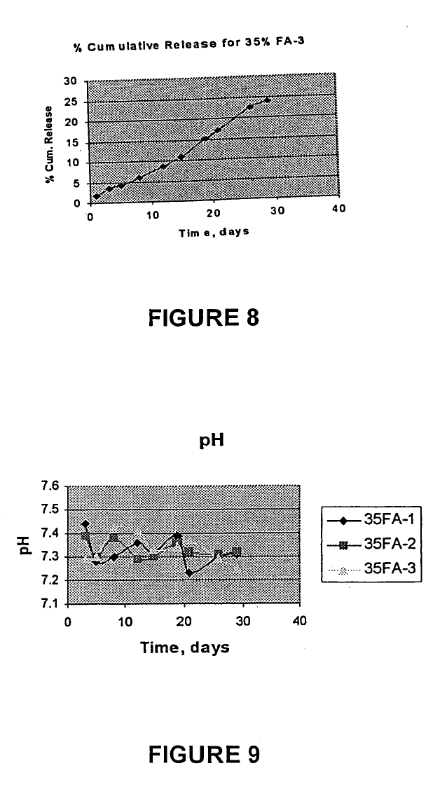

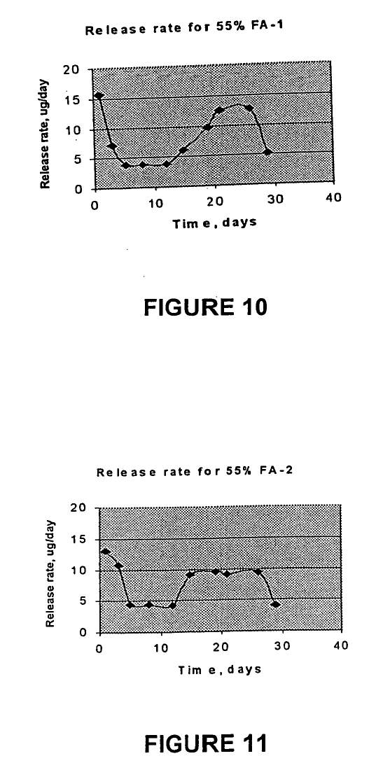

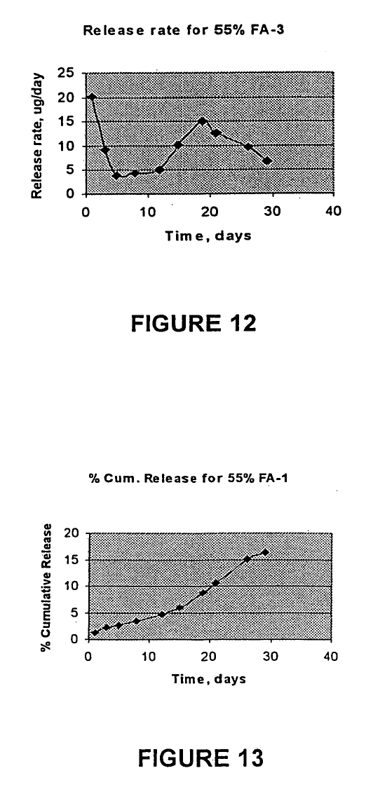

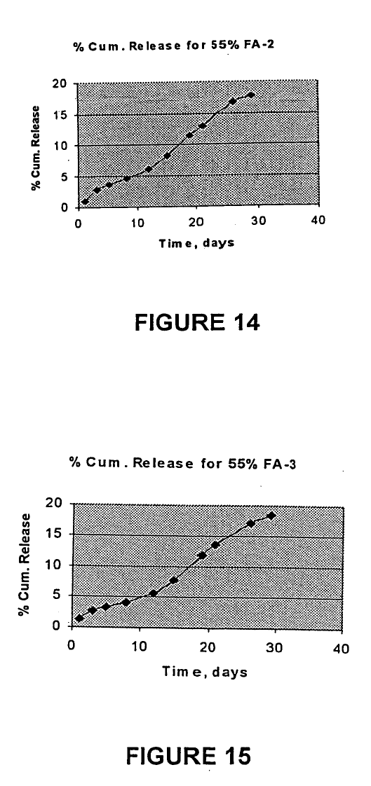

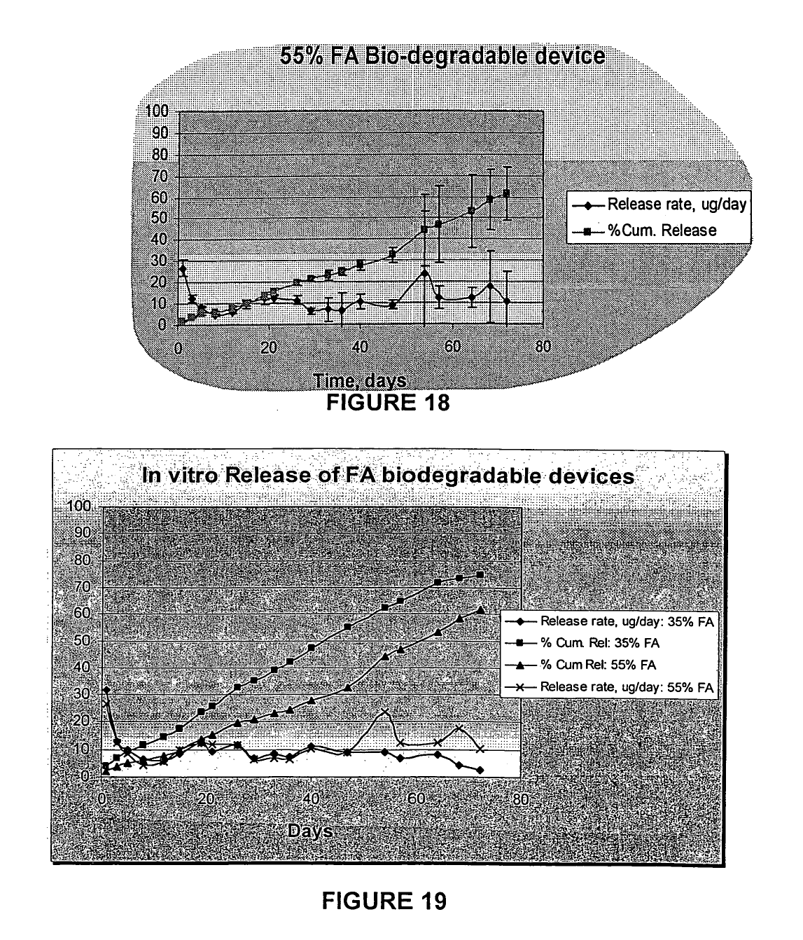

The present invention relates to novel chemical erosion controlled release drug delivery systems, produced from one or more biodegradable compositions such as but not limited to 50/50 poly(DL-lactide-co-glycolide) polymer (PLGA) and one or more hydrophobic or hydrophobically-enhanced pharmaceutical agents or drugs. By varying the hydrophobic or hydrophobically-enhanced pharmaceutical agent or drug load within a biodegradable composition, the overall biodegradable degradation rate of the delivery device and hence the drug release rate can be manipulated as desired. For example, several biodegradable chemical erosion controlled release drug delivery systems were prepared with 35 percent by weight and 55 percent by weight fluocinolone acetonide (FA) loads in 50/50 PLGA through an extrusion process. These drug delivery systems were capable of being inserted through a 0.55 mm diameter cannula used along with the 25-guage needle in the TSV Millenium™ vitrectomy system (Bausch & Lomb Incorporated, Rochester, N.Y.). An in vitro drug release study was conducted to determine the duration and the amount of drug released from the drug delivery systems as illustrated in FIGS. 3-5 and 10-12. Based on a thirty-day study, the 55 weight percent FA systems exhibited slower degradation due to increased hydrophobicity and consequently slower diffusion of the aqueous media resulting in a slower bioerodible degradation. After thirty days, the 35 percent by weight FA systems and the 55 percent by weight FA systems showed a cummulative release of about 25% and 17% respectively, as illustrated in FIGS. 6-8, 13-15, 17 and 18. In both cases, the FA release rate per day was at least approximately 5 μg. After seventy days, the 35 percent by weight FA systems and the 55 percent by weight FA systems showed a cumulative release of about 75% and 61% respectively, as illustrated in FIG. 19. Accordingly, the subject chemical erosion controlled release drug delivery systems allow for control of drug release rates based on the load of the hydrophobic or hydrophobically-enhanced drug to be delivered.

For purposes of the present invention, suitable biodegradable polymers for use in the subject chemical erosion controlled release drug delivery systems include for example but are not limited to poly(lactide)s, poly(glycolide)s, poly(lactide-co-glycolide)s, poly(lactic acid)s, poly(glycolic acid)s, poly(lactic acid-co-glycolic acid)s, polycaprolactones, polycarbonates, poly(ester amide)s, polyanhydrides, poly(amino acid)s, polyorthoesters, polyacetals, polycyanoacrylates, poly(ether ester)s, polydioxanones, poly(alkylene alkylate)s, copolymers of polyethylene glycol and polyorthoester, biodegradable polyurethanes, and blends and copolymers thereof.

For purposes of the present invention, suitable hydrophobic pharmaceutical agents or drugs for use in the subject chemical erosion controlled release drug delivery systems include any pharmaceutical agents or drugs that are hydrophobic, as defined herein as meaning sparingly soluble or slightly soluble in water, i.e., less than one percent drug/solution. Likewise, hydrophilic drugs or drugs having low hydrophobicity can be used in accordance with the present invention by increasing the hydrophobicity thereof. Such hydrophobicity-enhanced drugs are produced by admixing the hydrophilic drug or drug having low hydrophobicity with a suitable biocompatible hydrophobic agent. Suitable biocompatible hydrophobic agents include for example but are not limited to glycerol triacetate, glycerol diacetate, diethyl phthalate, dimethyl phthalate, phthalate esters, phosphate esters, fatty acid esters, glycerol derivatives, acetyl triethyl citrate, dibutyl tartrate and combinations thereof. Such hydrophobic agents influence drug release rate by filling the matrix polymer interstices. By filling the matrix polymer interstices, hydrophobic agents impede water diffusion into the bulk of the drug delivery system both by their hydrophobicity and by serving as physical blockages. Through the impediment of water diffusion, the hydrolytic degradation rate of the drug delivery system is reduced.

Suitable hydrophobic drugs, or drugs suitable upon hydrophobicity enhancement for use in the present invention include for example but are not limited to ametantrone, amphotericin B, annamycin, cyclosporin, daunorubicin, diazepam, doxorubicin, elliptinium, etoposide, fluocinolone acetonide, ketoconazole, methotrexate, miconazole, mitoxantrone, nystatin, phenytoin and vincristine. Other suitable pharmaceutically-active agents include but are not limited to cytokines and steroidal hormones for example estragenic, e.g., estradiol, and androgenic, e.g., testosterone, hormones, or other hormones that comprise a sterol backbone. Mixtures of more than one drug can also be incorporated into one drug delivery system for the purpose of co-administration.

Other pharmaceutically-active agents or drugs useful in the chemical erosion controlled release drug delivery system of the present invention include for example but are not limited to anti-glaucoma agents such as for example but not limited to intraocular pressure lowering agents such as for example diamox, neuroprotection agents such as for example nimodipine, beta blockers such as for example timolol maleate, betaxolol and metipranolol, mitotics such as for example pilocarpine, acetylcholine chloride, isofluorophate, demacarium bromide, echothiophateiodide, phospholine iodide, carbachol and physostigimine, epinephrine and salts such as for example dipivefrin hydrochloride, dichlorphenamide, acetazolamide and methazolamide; anti-diabetic edema agents such as for example but not limited to steroids such as for example fluocinolone, and anti-vascular endothelial growth factors (VEGF) receptors such as for example VEGF receptor tyrosine kinase inhibitors, pyrrolyl-methylene-indolinones and C₆₋₄₅ phenyl amino alkoxy quinazolines; anti-proliferative vitreoretinopathy agents such as for example but not limited to fluocinolone acetonide, dexamethasone, prednisolone and triamcinolone acetonide; anti-inflammatory agents such as for example but not limited to steroids such as for example hydrocortisone, hydrocortisone acetate, dexamethasone, fluocinolone, medrysone, methylprednisolone, prednisolone, prednisolone acetate, fluoromethalone, betamethasone and triamcinolone acetonide and immunological response modifiers such as for example cyclosporin; anti-ocular angiogenesis agents such as for example but not limited to anti VEGF receptors such as for example VEGF receptor tyrosine kinase inhibitors, pyrrolyl-methylene-indolinones and C₆₋₄₅ phenyl amino alkoxy quinazolines, anti-mobility agents such as for example cytochalasin B, steroids such as for example fluocinolone acetonide dexamethasone and prednisolone, matrix metalloproteinase (MMP) inhibitors such as for example benzodiazepine sulfonamide hydroxamic acids, and humanized antibodies, aptamers and peptides that are formulated to become sparingly soluble; antibiotics such as for example but not limited to ganciclovir; angiogenesis targeting agents such as for example but not limited to angiogenic growth factors such as for example VEGF, VEGF receptors, integrins, tissue factors, prostaglandin-cyclooxygenase 2 and MMPs; anti-cataract and anti-diabetic retinopathy agents such as for example but not limited to the aldose reductase inhibitors, tolrestat, lisinopril, enalapril and statil, thiol cross-linking agents, anticancer agents such as for example but not limited to retinoic acid, methotrexate, adriamycin, bleomycin, triamcinolone, mitomycin, cisplatinum, vincristine, vinblastine, actinomycin-D, ara-c, bisantrene, activated cytoxan, melphalan, mithramycin, procarbazine and tamoxifen, immune modulators, anti-clotting agents such as for example but not limited to tissue plasminogen activator, urokinase and streptokinase, anti-tissue damage agents such as for example but not limited to superoxide dismutase, proteins and nucleic acids such as for example but not limited to mono- and poly-clonal antibodies, enzymes, protein hormones and genes, gene fragments and plasmids, steroids, particularly anti-inflammatory or anti-fibrous agents such as for example but not limited to lodeprednol, etabonate, cortisone, hydrocortisone, prednisolone, prednisome, dexamethasone, progesterone-like compounds, medrysone (HMS) and fluorometholone, non-steroidal anti-inflammatory agents such as for example but not limited to ketrolac tromethamine, dichlofenac sodium and suprofen, antibiotics such as for example but not limited to loridine (cephaloridine), chloramphenicol, clindamycin, amikacin, tobramycin, methicillin, lincomycin, oxycillin, penicillin, amphotericin B, polymyxin B, cephalosporin family, ampicillin, bacitracin, carbenicillin, cepholothin, colistin, erythromycin, streptomycin, neomycin, sulfacetamide, vancomycin, silver nitrate, sulfisoxazole diolamine and tetracycline, other antipathogens including anti-viral agents such as for example but not limited to idoxuridine, trifluorouridine, vidarabine (adenine arabinoside), acyclovir (acycloguanosine), pyrimethamine, trisulfapyrimidine-2, clindamycin, nystatin, flucytosine, natamycin, and miconazole, piperazine derivatives such as for example but not limited to diethylcarbamazine, and cycloplegic and mydriatic agents such as for example but not limited to atropine, cyclogel, scopolamine, homatropine and mydriacyl.

Other suitable pharmaceutically-active agents or drugs include anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

Pharmaceutical agents or drugs of particular interest include hydrocortisone (5-20 mcg/l as plasma level), gentamycin (6-10 mcg/ml in serum), 5-fluorouracil (˜30 mg/kg body weight in serum), sorbinil, interleukin-2, phakan-a (a component of glutathione), thioloa-thiopronin, bendazac, acetylsalicylic acid, trifluorothymidine, interferon (α, β and γ), immune modulators such as for example but not limited to lymphokines and monokines and growth factors.

The drug hydrophobicity and load size within the drug delivery system dictates the rate of bioerodible degradation, and is a primary factor controlling the rate of drug release. Thus, by controlling the hydrophobicity of the drug and the drug load size within the drug delivery system, particular characteristics or properties are achieved. The particular characteristics or properties achieved may then be manipulated to achieve the desired rate of drug release. The desired rate of drug release may be determined based on the drug to be delivered, the location of delivery, the purpose of delivery and/or the therapeutic requirements of the individual patient.

The chemical erosion controlled release drug delivery systems of the present invention are described in still greater detail in the examples that follow.

### EXAMPLE 1

Chemical Erosion Controlled Release Drug Delivery System Sample Preparation and Study:

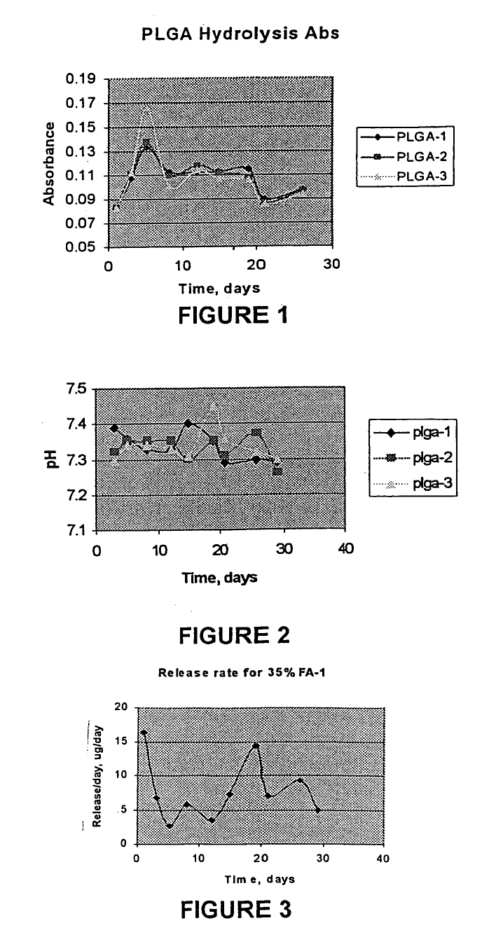

An Atlas™ lab mixing extruder (LME) (Dynisco Instruments, Franklin, Mass.) was used to mix and extrude PLGA/FA strands at 35 percent and 55 percent loadings and PLGA placebo filaments, each approximately 0.5 mm in diameter. These cylindrical filaments were stored in a dessicator unit. Three samples per loading approximately 0.55 mm diameter and 1 cm in length were cut, weighed and placed individually in a centrifuge tube containing 50 ml phosphate buffered solution, pH=7.4. Each sample was allowed to adhere to the wall of the centrifuge tube and placed on a rotating mixer at 8 revolutions per minute (rpm). All samples were then placed in an oven at 37° C. At periodic intervals, 15 ml solution samples from the 50 ml reservoir were removed,and replaced with equal volume of fresh phosphate buffered saline (PBS). The pH of the solution samples was measured. The solution samples were then diluted with 15 ml of fresh PBS and mixed thoroughly. The absorbance values were read on a UV/VIS spectrophotometer and peak values corresponding to glycolic acid and FA were read for each sample period as illustrated in FIG. 1. The release rate per day and percent cummulative release were determined.

50/50 DL-PLGA is an amorphous polymer. The primary pathway for PLGA biodegradation is through water diffusion into the polymer matrix, random hydrolysis, matrix fragmentation followed by extensive hydrolysis along with phagocytosis, diffusion and metabolism. For the first 30 days of the study, a transparent PLGA sample showed signs of increasing water diffusion as evidenced by the change in refractive index of the implant. No macro-fragmentation was visible. Other factors affecting the hydrolysis and consequently drug release are the surface area of the implant, polymer crystallinity and hydrophilicity as well as pH and temperature of the surrounding media. Extrusion of the polymer induces crystallinity which slows down degradation relative to other modes of fabrication such as compression molding or, to a lesser extent, injection molding. Molecular weight and glycolide content in the copolymer can also significantly affect the rate of hydrolysis as well as the mixing speed, rpm, of the tube tumbler. Peak absorbance values for glycolic acid show a relatively stable hydrolysis after an initial peak produced from surface diffusion. The system showed adequate buffering as seen by the narrow pH range measured over 30 days, as illustrated in FIG. 2.

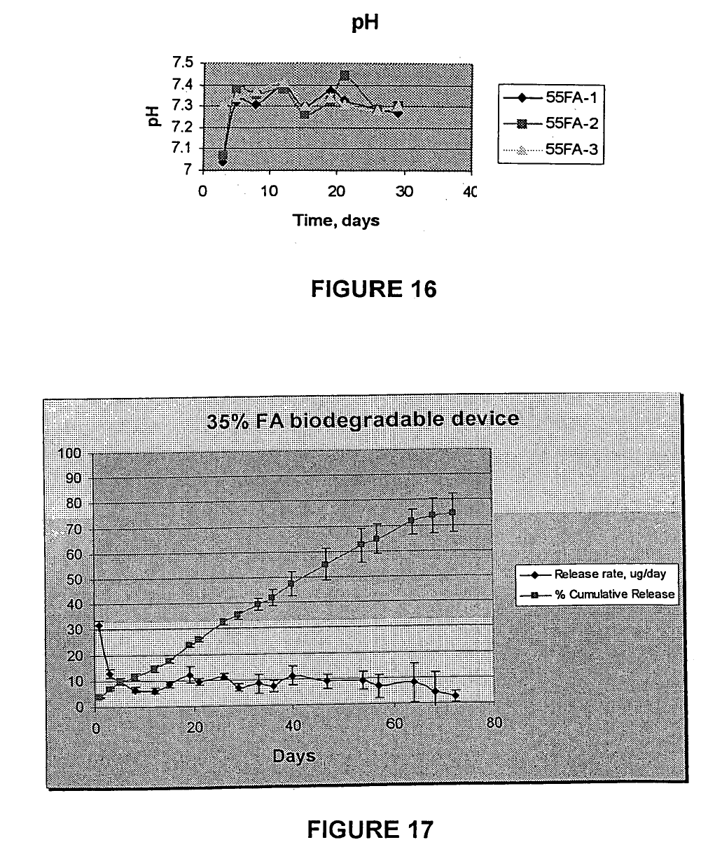

Presence of a hydrophobic compound, fluocinolone acetonide in PLGA significantly slows down the water diffusion rate as evidenced by the relatively smaller change in the size of the implant. The surface of the implant also appeared to be smoother than the PLGA implant. For the most part, the FA release rate exceeded 5 μg/day with a cumulative release of about 25 percent of the approximately 850 μg FA present in the implant. The system pH showed little change over the course of the 30 days, as illustrated in FIGS. 9 and 16, influenced by the slower PLGA hydrolysis and low acid constant, kₐ, for FA.

The 55 percent FA implants seem to be releasing at roughly the same rate as the 35 percent implant. The samples also appeared to be holding intact at the same level as the 35 percent implants. The pH of the system seems to be well buffered as well.

In conclusion, similar release rates per day were observed for both 35 percent and 55 percent FA implants during the first 30 days of study, which seems to be primarily a diffusion controlled process. The percent cumulative release of FA, based on estimated FA loading, observed so far is significantly less for the 55 percent implants relative to the 35 percent implants.

Chemical erosion controlled release drug delivery systems of the present invention may be manufactured in any shape or size suitable for the intended purpose for which they are intended to be used. For example, for use as an inner back of the eye implant, the subject chemical erosion controlled release drug delivery system would preferably be no larger in size than 3 mm². Methods of manufacturing the subject chemical erosion controlled release drug delivery systems include cast molding, extrusion, and like methods known to those skilled in the art. Once manufactured, the subject chemical erosion controlled release drug delivery systems are packaged and sterilized using customary methods known to those skilled in the art.

Chemical erosion controlled release drug delivery systems of the present invention may be used in a broad range of therapeutic applications. In the field of ophthalmology for example, the subject controlled release drug delivery system is used by implantation within the interior portion of an eye. However, the subject chemical erosion controlled release drug delivery system may likewise be used in accordance with other surgical procedures known to those skilled in the field of ophthalmology.

While there is shown and described herein chemical erosion controlled release drug delivery systems and methods of making and using the same, it will be manifest to those skilled in the art that various modifications may be made without departing from the spirit and scope of the underlying inventive concept. The present invention is likewise not intended to be limited to particular monomers, copolymers and systems described herein except insofar as indicated by the scope of the appended claims.

1. A chemical erosion controlled drug delivery system comprising:
a mixture of a biodegradable polymer and a hydrophobic pharmaceutically-active agent in a therapeutically effective amount wherein the drug delivery system has a selected concentration of the pharmaceutically-active agent such that when the drug delivery system is compared to a comparative system with an incrementally lower concentration of the pharmaceutically-active agent, the drug delivery system (i) has a release rate for the pharmaceutically-active agent that is less than proportionally higher, the same or lower than a comparative system and/or (ii) has a duration of release of the pharmaceutically-active agent that is the same or longer than the comparative system.

2. The drug delivery system of claim 1, wherein the system is sized and configured to be inserted into the ocular region of a human patient.

3. The drug delivery system of claim 2, wherein the system is sized and configured to be inserted into the posterior segment of the eye of a human patient.

4. The drug delivery system of claim 1, wherein the system is configured to be inserted into the vitreous of the eye of a human patient.

5. The drug delivery system of claim 1, wherein the mixture consists essentially of biodegradable polymer and a therapeutically effective amount of hydrophobic pharmaceutically-active agent.

6. The drug delivery system of claim 1, wherein the system occupies a maximum volume of about 26 mm³.

7. The drug delivery system of claim 1, wherein the system has a maximum mass of about 50 mg.

8. The drug delivery system of claim 1, wherein the system has a maximum amount of the pharmaceutically-active agent of about 25 mg.

9. The drug delivery system of claim 1, wherein said at least one pharmaceutically-active agent is selected from the group consisting of cytokines, tyrosine kinase inhibitors and steroidal hormones.

10. The drug delivery system of claim 1, wherein said at least one pharmaceutically-active agent is selected from the group consisting of anti-glaucoma agents, neuroprotection agents, beta blockers, mitotics, epinephrine, anti-diabetic edema agents, vascular endothelial growth factor (VEGF) antagonists, tyrosine kinase inhibitors, pyrrolyl-methylene-indolinones, C₆₋₄₅ phenyl amino alkoxy quinazolines, anti-proliferative vitreoretinopathy agents, anti-inflammatory agents, immunological response modifiers, anti-ocular angiogenesis agents, anti-mobility agents, steroids, matrix metalloproteinase (MMP) inhibitors, humanized antibodies, aptamers, peptides, antibiotics, angiogenesis targeting agents, anti-cataract and anti-diabetic retinopathy agents, thiol cross-linking agents, anticancer agents, immune modulators, anti-clotting agents, anti-tissue damage agents, proteins, nucleic acids, anti-fibrous agents, non-steroidal anti-inflammatory agents, antibiotics, antipathogens, piperazine derivatives, cycloplegic and mydriatic agents anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

11. The drug delivery system of claim 1, wherein said biodegradable polymer is selected from the group consisting of poly(lactide)s, poly(glycolide)s, poly(lactide-co-glycolide)s, poly(lactic acid)s, poly(lactic acid-co-glycolic acid)s, polycaprolactones, polycarbonates, poly(ester amide)s, polyanhydrides, poly(amino acid)s, polyorthoesters, polyacetals, polycyanoacrylates, poly(ether ester)s, polydioxanones, poly(alkylene alkylate)s, copolymers of poly(ethylene glycol) and polyorthoesters, biodegradable polyurethanes and blends and copolymers thereof.

12. The drug delivery system of claim 1, wherein the biodegradable polymer is poly(lactic acid-co-glycolic acid)s.

13. The drug delivery system of claim 1, wherein the biodegradable polymer has a ratio of lactic acid to glycolic acid that is a minimum of 0.1 and a maximum of about 10.

14. The drug delivery system of claim 1, wherein the biodegradable polymer has a ratio of poly(lactic-co-glycolic) acid to the pharmaceutically-active agent that is a minimum of about is a minimum of about 0.8 and a maximum of about 4.

15. The drug delivery system of claim 1, wherein the mixture comprises a hydrophobic agent.

16. The drug delivery system of claim 1, wherein the mixture further comprises a hydrophobic agent that has a solubility greater than 90 μg/ml in a buffered saline solution at 25° C.

17. The drug delivery system of claim 1, wherein the drug delivery device delivers a minimum of about 0.1 μg is released over a minimum period of 3 weeks.

18. The drug delivery system of claim 1, wherein the hydrophobic pharmaceutically-active agent has a solubility that is less than about 90 μg/ml in a buffered saline solution at 25° C.

19. The drug delivery system of claim 1, wherein the incrementally lower concentration is 1% lower than the selected concentration and the drug delivery system (i) has a release rate for the pharmaceutically-active agent that is no more than about 0.9% higher, the same or lower than a comparative system.

20. A drug delivery device comprising:
a matrix of a biodegradable polymer and a hydrophobic pharmaceutically-active agent in a therapeutically effective amount wherein the drug delivery device has a selected concentration of the pharmaceutically-active agent such that when the drug delivery device is compared to a comparative device with an incrementally lower concentration of the pharmaceutically-active agent, the drug delivery device has a release rate for the pharmaceutically-active agent that is less than proportionally higher, the same or lower than a comparative device.

21. The drug delivery device of claim 20, wherein the device is sized and configured to be implanted into the ocular region of a human patient.

22. The drug delivery device of claim 21, wherein the matrix consists essentially of biodegradable polymer and a therapeutically effective amount of hydrophobic pharmaceutically-active agent.

23. The drug delivery device of claim 21, wherein the device has a maximum mass of about 50 mg.

24. The drug delivery device of claim 23, wherein said at least one pharmaceutically-active agent is selected from the group consisting of anti-glaucoma agents, neuroprotection agents, beta blockers, mitotics, epinephrine, anti-diabetic edema agents, vascular endothelial growth factor (VEGF) antagonists, tyrosine kinase inhibitors, pyrrolyl-methylene-indolinones, C₆₋₄₅ phenyl amino alkoxy quinazolines, anti-proliferative vitreoretinopathy agents, anti-inflammatory agents, immunological response modifiers, anti-ocular angiogenesis agents, anti-mobility agents, steroids, matrix metalloproteinase (MMP) inhibitors, humanized antibodies, aptamers, peptides, antibiotics, angiogenesis targeting agents, anti-cataract and anti-diabetic retinopathy agents, thiol cross-linking agents, anticancer agents, immune modulators, anti-clotting agents, anti-tissue damage agents, proteins, nucleic acids, anti-fibrous agents, non-steroidal anti-inflammatory agents, antibiotics, antipathogens, piperazine derivatives, cycloplegic and mydriatic agents anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

25. The drug delivery device of claim 23, wherein said biodegradable polymer is selected from the group consisting of poly(lactide)s, poly(glycolide)s, poly(lactide-co-glycolide)s, poly(lactic acid)s, poly(lactic acid-co-glycolic acid)s, polycaprolactones, polycarbonates, poly(ester amide)s, polyanhydrides, poly(amino acid)s, polyorthoesters, polyacetals, polycyanoacrylates, poly(ether ester)s, polydioxanones, poly(alkylene alkylate)s, copolymers of poly(ethylene glycol) and polyorthoesters, biodegradable polyurethanes and blends and copolymers thereof.

26. The drug delivery device of claim 25, wherein the biodegradable polymer is poly(lactic acid-co-glycolic acid)s.

27. The drug delivery device of claim 23, wherein the drug delivery device delivers a minimum of about 0.1 μg is released over a minimum period of 3 weeks.

28. The drug delivery device of claim 23, wherein the hydrophobic pharmaceutically-active agent has a solubility that is less than about 90 μg/ml in a buffered saline solution at 25° C.

29. A drug delivery device comprising:
a matrix of a biodegradable polymer and a hydrophobic pharmaceutically-active agent in a therapeutically effective amount wherein the drug delivery device has a selected concentration of the pharmaceutically-active agent such that when the drug delivery device is compared to a comparative device with an incrementally lower concentration of the pharmaceutically-active agent, the drug delivery device has a duration of release of the pharmaceutically-active agent that is the same or longer than the comparable device.

30. The drug delivery device of claim 29, wherein the device is sized and configured to be implanted into the ocular region of a human patient.

31. The drug delivery device of claim 30, wherein the matrix consists essentially of biodegradable polymer and a therapeutically effective amount of hydrophobic pharmaceutically-active agent.

32. The drug delivery device of claim 30, wherein the device has a maximum mass of about 50 mg.

33. The drug delivery device of claim 32, wherein said at least one pharmaceutically-active agent is selected from the group consisting of anti-glaucoma agents, neuroprotection agents, beta blockers, mitotics, epinephrine, anti-diabetic edema agents, vascular endothelial growth factor (VEGF) antagonists, tyrosine kinase inhibitors, pyrrolyl-methylene-indolinones, C₆₋₄₅ phenyl amino alkoxy quinazolines, anti-proliferative vitreoretinopathy agents, anti-inflammatory agents, immunological response modifiers, anti-ocular angiogenesis agents, anti-mobility agents, steroids, matrix metalloproteinase (MMP) inhibitors, humanized antibodies, aptamers, peptides, antibiotics, angiogenesis targeting agents, anti-cataract and anti-diabetic retinopathy agents, thiol cross-linking agents, anticancer agents, immune modulators, anti-clotting agents, anti-tissue damage agents, proteins, nucleic acids, anti-fibrous agents, non-steroidal anti-inflammatory agents, antibiotics, antipathogens, piperazine derivatives, cycloplegic and mydriatic agents anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

34. The drug delivery device of claim 32, wherein said biodegradable polymer is selected from the group consisting of poly(lactide)s, poly(glycolide)s, poly(lactide-co-glycolide)s, poly(lactic acid)s, poly(lactic acid-co-glycolic acid)s, polycaprolactones, polycarbonates, poly(ester amide)s, polyanhydrides, poly(amino acid)s, polyorthoesters, polyacetals, polycyanoacrylates, poly(ether ester)s, polydioxanones, poly(alkylene alkylate)s, copolymers of poly(ethylene glycol) and polyorthoesters, biodegradable polyurethanes and blends and copolymers thereof.

35. The drug delivery device of claim 34, wherein the biodegradable polymer is poly(lactic acid-co-glycolic acid)s.

36. The drug delivery device of claim 34, the drug delivery device delivers a minimum of about 0.1 μg is released over a minimum period of 3 weeks.

37. The drug delivery device of claim 34, wherein the hydrophobic pharmaceutically-active agent has a solubility that is less than about 90 μg/ml in a buffered saline solution at 25° C.

38. A drug delivery device comprising:
a matrix of a biodegradable polymer and a hydrophobic pharmaceutically-active agent in a therapeutically effective amount, wherein the hydrophobic pharmaceutically-active agent has a solubility that is less than about 90 μg/ml in a buffered saline solution at 25° C.

39. The drug delivery device of claim 38, wherein the device is sized and configured to be inserted into the ocular region of a human patient.

40. The drug delivery device of claim 39, wherein the device is sized and configured to be inserted into the posterior segment of the eye of a human patient.

41. The drug delivery device of claim 39, wherein the device is sized and configured to be inserted into the vitreous of the eye of a human patient.

42. The drug delivery device of claim 39, wherein the matrix consists essentially of biodegradable polymer and a therapeutically effective amount of hydrophobic pharmaceutically-active agent.

43. The drug delivery device of claim 39, wherein the device occupies a maximum volume of about 26 mm³.

44. The drug delivery device of claim 39, wherein the device has a maximum mass of about 50 mg.

45. The drug delivery device of claim 39, wherein the device has a maximum amount of the pharmaceutically-active agent of about 25 mg.

46. The drug delivery device of claim 39, wherein said at least one pharmaceutically-active agent is selected from the group consisting of cytokines, tyrosine kinase inhibitors and steroidal hormones.

47. The drug delivery device of claim 39, wherein said at least one pharmaceutically-active agent is selected from the group consisting of anti-glaucoma agents, neuroprotection agents, beta blockers, mitotics, epinephrine, anti-diabetic edema agents, vascular endothelial growth factor (VEGF) antagonists, pyrrolyl-methylene-indolinones, C₆₋₄₅ phenyl amino alkoxy quinazolines, anti-proliferative vitreoretinopathy agents, anti-inflammatory agents, immunological response modifiers, anti-ocular angiogenesis agents, anti-mobility agents, steroids, matrix metalloproteinase (MMP) inhibitors, humanized antibodies, aptamers, peptides, antibiotics, angiogenesis targeting agents, anti-cataract and anti-diabetic retinopathy agents, thiol cross-linking agents, anticancer agents, immune modulators, anti-clotting agents, anti-tissue damage agents, proteins, nucleic acids, anti-fibrous agents, non-steroidal anti-inflammatory agents, antibiotics, antipathogens, piperazine derivatives, cycloplegic and mydriatic agents anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

48. The drug delivery device of claim 39, wherein said biodegradable polymer is selected from the group consisting of poly(lactide)s, poly(glycolide)s, poly(lactide-co-glycolide)s, poly(lactic acid)s, poly(lactic acid-co-glycolic acid)s, polycaprolactones, polycarbonates, poly(ester amide)s, polyanhydrides, poly(amino acid)s, polyorthoesters, polyacetals, polycyanoacrylates, poly(ether ester)s, polydioxanones, poly(alkylene alkylate)s, copolymers of poly(ethylene glycol) and polyorthoesters, biodegradable polyurethanes and blends and copolymers thereof.

49. The drug delivery device of claim 39, wherein the biodegradable polymer is poly(lactic acid-co-glycolic acid)s.

50. The drug delivery device of claim 39, wherein the biodegradable polymer has a ratio of lactic acid to glycolic acid that is a minimum of about 0.1 and a maximum of about 10.

51. The drug delivery device of claim 39, wherein the biodegradable polymer has a ratio of poly(lactic-co-glycolic) acid to the pharmaceutically-active agent that is a minimum of about is a minimum of about 0.8 and a maximum of about 4.

52. The drug delivery device of claim 39, the drug delivery device delivers a minimum of about 0.1 μg is released over a minimum period of 3 weeks.

53. The drug delivery device of claim 39, wherein the active agent has a selected concentration such that a 1% increase in concentration results in an increase in the duration of release that is a minimum of about 0.1%.

54. The drug delivery device of claim 39, wherein the active agent has a selected concentration such that a 1% increase in concentration results in a decrease, no change or an increase in the delivery rate that is a maximum of about 0.9%.

55. A chemical erosion controlled drug delivery device comprising:
a therapeutic mixture of a biodegradable polymer and a minimum amount of about 45 wt. % of a pharmaceutically-active agent based upon the total weight of the biodegradable polymer and the pharmaceutically-active agent, wherein the pharmaceutically-active agent is characterized in that a 55 wt. % mixture of the pharmaceutically-active agent in a PLGA test matrix releases no more than about 70 wt % of the pharmaceutically-active agent in a three-week period and that the cumulative release rate of the 55 wt. % mixture of the hydrophobic pharmaceutically-active agent in a PLGA test matrix is not more than about 10% greater than the cumulative release rate of a 35 wt. % mixture of the pharmaceutically-active agent in a test matrix over a three-week test period.

56. The drug delivery device of claim 55, wherein the device is sized and configured to be inserted into the ocular region of a human patient.

57. The drug delivery device of claim 56, wherein the device is sized and configured to be inserted into the posterior segment of the eye of a human patient.

58. The drug delivery device of claim 56, wherein the device has a minimum amount of about 50 wt. % of a pharmaceutically-active agent based upon the total weight of the biodegradable polymer and the pharmaceutically-active agent.

59. The drug delivery device of claim 56, wherein a 55 wt. % mixture of the pharmaceutically-active agent in a PLGA test matrix releases no more than about 60 wt % of the pharmaceutically-active agent in a three-week period.

60. The drug delivery device of claim 56, wherein the cumulative release rate of the 55 wt. % mixture of the hydrophobic pharmaceutically-active agent in a PLGA test matrix is not more than about 5% greater than the cumulative release rate of a 35 wt. % mixture of the pharmaceutically-active agent in a test matrix over a three-week test period.

61. The drug delivery device of claim 56, wherein the device is sized and configured to be inserted in the vitreous of the eye of a human patient.

62. The drug delivery device of claim 56, wherein the therapeutic mixture consists essentially of biodegradable polymer and a therapeutically effective amount of hydrophobic pharmaceutically-active agent.

63. The drug delivery device of claim 56, wherein the device has a maximum mass of about 50 mg.

64. The drug delivery device of claim 56, wherein said at least one pharmaceutically-active agent is selected from the group consisting of anti-glaucoma agents, neuroprotection agents, beta blockers, mitotics, epinephrine, anti-diabetic edema agents, vascular endothelial growth factor (VEGF) antagonists, pyrrolyl-methylene-indolinones, C₆₋₄₅ phenyl amino alkoxy quinazolines, anti-proliferative vitreoretinopathy agents, anti-inflammatory agents, immunological response modifiers, anti-ocular angiogenesis agents, anti-mobility agents, steroids, matrix metalloproteinase (MMP) inhibitors, humanized antibodies, aptamers, peptides, antibiotics, angiogenesis targeting agents, anti-cataract and anti-diabetic retinopathy agents, thiol cross-linking agents, anticancer agents, immune modulators, anti-clotting agents, anti-tissue damage agents, proteins, nucleic acids, anti-fibrous agents, non-steroidal anti-inflammatory agents, antibiotics, antipathogens, piperazine derivatives, cycloplegic and mydriatic agents anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

65. The drug delivery device of claim 56, wherein said biodegradable polymer is selected from the group consisting of poly(lactide)s, poly(glycolide)s, poly(lactide-co-glycolide)s, poly(lactic acid)s, poly(lactic acid-co-glycolic acid)s, polycaprolactones, polycarbonates, poly(ester amide)s, polyanhydrides, poly(amino acid)s, polyorthoesters, polyacetals, polycyanoacrylates, poly(ether ester)s, polydioxanones, poly(alkylene alkylate)s, copolymers of poly(ethylene glycol) and polyorthoesters, biodegradable polyurethanes and blends and copolymers thereof.

66. The drug delivery device of claim 56, wherein the biodegradable polymer is poly(lactic acid-co-glycolic acid)s.

67. The drug delivery device of claim 66, wherein the biodegradable polymer has a ratio of poly(lactic-co-glycolic) acid to the pharmaceutically-active agent that is a minimum of about is a minimum of about 0.8 and a maximum of about 4.

68. The drug delivery device of claim 56, wherein the hydrophobic pharmaceutically-active agent has a solubility that is less than about 90 μg/ml in a buffered saline solution at 25° C.

69. A chemical erosion controlled drug delivery system comprising:
a biodegradable polymer; and
a hydrophobic pharmaceutically-active agent selected from the group consisting of ametantrone, amphotericin B, annamycin, cyclosporin, daunorubicin, diazepam, doxorubicin, elliptinium, etoposide, fluocinolone acetonide, ketoconazole, methotrexate, miconazole, mitoxantrone, nystatin, phenytoin, lodeprednol, triamcinolone acetonide and vincristine in a therapeutically effective amount wherein the drug delivery system has a selected concentration of the pharmaceutically-active agent such that when the drug delivery system is compared to a comparative system with an incrementally lower concentration of the pharmaceutically-active agent, the drug delivery system (i) has a release rate for the pharmaceutically-active agent that is less than proportionally higher, the same or lower than a comparative system and/or (ii) has a duration of release of the pharmaceutically-active agent that is the same or longer than the comparative system.

70. A method of making the system of claims 1 or 69 comprising:
encapsulating in a biodegradable polymer a therapeutically effective amount of at least one pharmaceutically-active agent, wherein the drug delivery system is sized and configured to be inserted into the eye of a patient.

71. A method of making the device of claims 20, 29, 38 or 55 comprising:
encapsulating in a biodegradable polymer a therapeutically effective amount of at least one pharmaceutically-active agent, wherein the drug delivery system is sized and configured to be inserted into the eye of a patient.

73. A method of making the system of claims 1 or 69 comprising:
mixing in a biodegradable polymer a therapeutically effective amount of at least one pharmaceutically-active agent, wherein the drug delivery system is sized and configured to be inserted into the eye of a patient.

74. A method of making the device of claims 20, 29, 38 or 55 comprising:
mixing in a biodegradable polymer a therapeutically effective amount of at least one pharmaceutically-active agent, wherein the drug delivery system is sized and configured to be inserted into the eye of a patient.

75. A method of using the system of claims 1 or 69 comprising:
creating an incision within an eye; and
implanting the system within said eye through said incision.

76. A method of using the system of claim 1 or 69 comprising:
creating an incision within an eye; and
implanting the system within said eye through said incision using a cannula used along with a needle of a vitrectomy system.

77. A method of using the device of claims 20, 29, 38 or 55 comprising:
creating an incision within an eye; and
implanting the device within said eye through said incision.

78. A method of using the device of claims 20, 29, 38 or 55 comprising:
creating an incision within an eye; and
implanting the device within said eye through said incision using a cannula used along with a needle of a vitrectomy system.

79. A method of making a drug delivery device configured to deliver a pharmaceutically active agent to a patient comprising:
mixing a biodegradable polymer and a pharmaceutically active agent into a mixture wherein the amount of biodegradable polymer is adjusted to a higher concentration of pharmaceutically active agent in the mixture and further resulting in a longer release profile; and
forming the mixture into a drug delivery device.

80. The method of claim 79, wherein the drug delivery device is sized and configured to be inserted into the ocular region of a human patient.

81. The method of claim 80, wherein the drug delivery device is sized and configured to be inserted into the posterior segment of the eye of a human patient.

82. The method of claim 81, wherein the device is sized and configured to be inserted into the vitreous of the eye of a human patient.

83. The method of claim 79, wherein the mixture consists essentially of biodegradable polymer and a therapeutically effective amount of hydrophobic pharmaceutically-active agent.

84. The method of claim 79, wherein the drug delivery device occupies a maximum volume of about 26 mm³.

85. The method of claim 79, wherein the drug delivery device has a maximum mass of about 50 mg.

86. The method of claim 79, wherein the drug delivery device has a maximum amount of the pharmaceutically-active agent of about 25 mg.

87. The method of claim 79, wherein said at least one pharmaceutically-active agent is selected from the group consisting of cytokines, tyrosine kinase inhibitors and steroidal hormones.

88. The method of claim 79, wherein said at least one pharmaceutically-active agent is selected from the group consisting of anti-glaucoma agents, neuroprotection agents, beta blockers, mitotics, epinephrine, anti-diabetic edema agents, vascular endothelial growth factor (VEGF) antagonists, tyrosine kinase inhibitors, pyrrolyl-methylene-indolinones, C₆₋₄₅ phenyl amino alkoxy quinazolines, anti-proliferative vitreoretinopathy agents, anti-inflammatory agents, immunological response modifiers, anti-ocular angiogenesis agents, anti-mobility agents, steroids, matrix metalloproteinase (MMP) inhibitors, humanized antibodies, aptamers, peptides, antibiotics, angiogenesis targeting agents, anti-cataract and anti-diabetic retinopathy agents, thiol cross-linking agents, anticancer agents, immune modulators, anti-clotting agents, anti-tissue damage agents, proteins, nucleic acids, anti-fibrous agents, non-steroidal anti-inflammatory agents, antibiotics, antipathogens, piperazine derivatives, cycloplegic and mydriatic agents anticholinergics, anticoagulants, antifibrinolytics, antihistamines, antimalarials, antitoxins, chelating agents, hormones, immunosuppressives, thrombolytics, vitamins, salts, desensitizers, prostaglandins, amino acids, metabolites and antiallergenics.

89. The method of claim 79, wherein said biodegradable polymer is selected from the group consisting of poly(lactic acid), poly(lactic acid-co-glycolic acid), polycaprolactones, polycarbonates, poly(ester amide), polyanhydrides, poly(amino acid), polyorthoesters, polyacetals, polycyanoacrylates, poly(ether ester), polydioxanones, poly(alkylene alkylate), copolymers of poly(ethylene glycol) and polyorthoesters, biodegradable polyurethanes and blends and copolymers thereof.

90. The method of claim 79, wherein the biodegradable polymer is poly(lactic acid-co-glycolic acid).

91. The method of claim 79, wherein the biodegradable polymer has a ratio of lactic acid to glycolic acid that is a minimum of 0.1 and a maximum of about 10.

92. The method of claim 79, wherein the biodegradable polymer has a ratio of poly(lactic-co-glycolic acid) to the pharmaceutically-active agent that is a minimum of about is a minimum of about 0.8 and a maximum of about 4.

93. The method of claim 79, wherein the mixture comprises a hydrophobic agent.

94. The method of claim 79, wherein the mixture further comprises a hydrophobic agent that has a solubility greater than 90 μg/ml in a buffered saline solution at 25° C.

95. The method of claim 79, wherein the drug delivery device delivers a minimum of about 0.1 μg of pharmaceutically active agent over a minimum period of 3 weeks.

96. The method of claim 79, wherein the hydrophobic pharmaceutically-active agent has a solubility that is less than about 90 μg/ml in a buffered saline solution at 25° C.

97. A drug delivery device that is made by the method of claim 79.
